(19) United States
(12) Patent Application Publication
(10) Pub. No.: US 2024/0156726 A1
(43) Pub. Date: May 16, 2024

(54) MULTI-LAYER ORAL THIN FILM

(71) Applicant: LTS LOHMANN THERAPIE-SYSTEME AG, Andemach (DE)

(72) Inventors: Michael LINN, Waldböckelheim (DE); Mario FICKER, Bonn (DE); Claudia NORELLI, Hillscheid (DE)

(21) Appl. No.: 18/272,189

(22) PCT Filed: Jan. 14, 2022
(86) PCT No.: PCT/EP2022/050791

§ 371 Date: Jul. 13, 2023

(30) Foreign Application Priority Data
Jan. 15, 2021 (DE) 10 2021 100 779.8

(51) Int. Cl. A61K 9/00; A61K 31/135; A61K 47/10

(52) U.S. Cl. CPC A61K 9/006; A61K 31/135; A61K 47/10

(57) ABSTRACT

The present invention relates to a multi-layer oral thin film comprising a matrix layer, which contains at least one polymer and at least one pharmaceutically active agent, and at least one backing layer, wherein the at least one backing layer comprises at least one polyethylene glycol, to a method for production of the oral thin film, and to the use of such an oral thin film as a medicament.

The present invention relates to a multi-layer oral thin film, to a method for production thereof, and to the use of such an oral thin film as a medicament.

Oral thin films are thin films containing at least one pharmaceutically active agent that are placed directly in the oral cavity or against the oral mucosa and dissolve or macerate there and in so doing deliver the active agent. These films are, especially, thin, one- or multi-layer, active-agent-containing polymer-based films which, when applied to a mucous membrane, especially the oral mucosa, can deliver the active agent directly into same. The very good blood supply to the oral mucosa ensures a rapid transfer of the active agent into the bloodstream. This dosage system has the advantage that the active agent is absorbed for the most part by the mucous membrane, thus avoiding the first-pass effect, which occurs in the case of the conventional dosage form of an active agent in tablet form. The active agent may be dissolved, emulsified or dispersed in the film.

Oral thin films known from the prior art have the disadvantage that if they are intended to remain for a longer time at a point on the mucous membrane of a patient, they are exposed to a permanent erosion. This leads to a large part of the material being swallowed and thus not remaining at the application site for the desired duration. However, the residence time may well be of decisive importance for the transmucosal transport of the pharmaceutically active agent. Furthermore, many active agents have a bad taste and are perceived as unpleasant, especially when they come into contact with the tongue.

A protective layer on the rear side can prevent liquid from penetrating the formulation and dissolving it too quickly, so that the active agent remains at the application site for the maximum time to achieve the greatest possible permeation through the mucosa or a delayed release. Another effect of the backing layer is that it prevents the administered film from detaching from the application site and adhering elsewhere, such as the teeth. Furthermore, the bad taste caused by the active agent can be concealed by a backing layer.

Insoluble or slowly soluble polymers or polymer films are often used as material for such backing layers. These have the disadvantage, however, that they have to be removed or swallowed following the application.

Backing layers made of slowly soluble polymers also have the disadvantage that they are based on long-chain, high-molecular polymers. These are difficult to process due to their high viscosity (long drying times, irregular films). Furthermore, they tend to increase the viscosity of the saliva in the oral cavity, resulting in a slimy feeling.

A further problem is the often inadequate bonding between the parts constituted by the active-agent-containing layer and the backing layer. There is a risk that both parts will detach from one another during storage if, for example, they react differently to air humidity.

The aim of the present invention lies in overcoming the above-mentioned disadvantages of the prior art. Especially, it is the aim of the present invention to provide a multi-layer oral thin film having at least one backing layer, wherein the backing layer has especially a low melting point, a safe toxicity profile, and a smooth surface. Furthermore, the backing layer should dissolve as slowly as possible and thus protect the applied film from being swallowed and protect the tongue against direct contact with the active agent, thus preventing or reducing an unpleasant taste resulting from the active agent. The active agent should also be protected from additional saliva, which is advantageous especially if the active agent or the active agent permeation is pH-sensitive and the pH value would change as a result of too much saliva.

Furthermore, a production method for such a film is to be provided, which ensures a fixed connection in the oral thin film between the active-agent-containing layer and the backing layer.

The above aim is addressed by a multi-layer oral thin film according to claim 1, especially by a multi-layer oral thin film comprising a matrix layer, which contains at least one polymer and at least one pharmaceutically active agent, and at least one backing layer, wherein the at least one backing layer comprises at least one polyethylene glycol.

Such a multi-layer oral thin film has the advantage that, on account of its smooth surface, low melting point and safe toxicity profile, the layer comprising a polyethylene glycol is well suited as a backing layer for oral thin films. The layer comprising a polyethylene glycol dissolves slowly in the mouth (more slowly than the active agent layer) and thus protects the applied oral thin film from being swallowed and additionally protects the tongue against direct contact with the active agent, which prevents or reduces an unpleasant taste resulting from the active agent. Furthermore, the active agent is protected from additional saliva, which is advantageous for example if the active agent or the active agent permeation is pH-sensitive and the pH value would change as a result of too much saliva. The different roughness of the two layers (the layer comprising a polyethylene glycol is rather smooth and the active-agent-containing layer is rather rough by comparison) can be used by the patient to determine which side of the oral thin film is applied towards the mucosa. Due to the different roughness, the patient also knows, as a result of the feel in their mouth, whether the oral thin film is the right side up in the mouth.

In addition, the backing layer can be coloured with colouring agent to achieve better visibility.

Furthermore, it is possible to incorporate flavourings to improve the mouthfeel during application, which is a major advantage over known films.

In the present document, the word “comprising” can also mean “consisting of”.

The term “backing layer” is understood to mean a layer of the multi-layer oral thin film that is one of the outermost layers of the multi-layer oral thin film.

Polyethylene glycols (PEG) are compounds of the general formula:

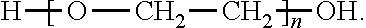

Higher molecular solid polyethylene glycols (melting temperature about 65° C.) are often also called polyethylene oxides or polyoxyethylenes (abbreviated to PEO or, more rarely, PEOX) or polywaxes. In this document, the terms “polyethylene glycol”, “polyethylene oxide” and “polyox” are used equivalently.

The multi-layer oral thin film according to the invention is preferably characterised in that the matrix layer comprises at least one water-soluble polymer.

Water-soluble polymers comprise chemically very different natural or synthetic polymers, the common feature of which is their solubility in water or aqueous media. A precondition is that these polymers have a number of hydrophilic groups sufficient for the water solubility and are not crosslinked. The hydrophilic groups may be non-ionic, anionic, cationic and/or zwitterionic.

Water-soluble polymers preferably have a solubility in water of greater than 100 g/L at 25° C.

The at least one water-soluble polymer is preferably selected from the group consisting of starch and starch derivatives, dextrans, cellulose derivatives, such as carboxymethyl cellulose, hydroxypropyl cellulose, hydroxyethyl cellulose, hydroxypropyl methylcellulose, hydroxypropyl ethyl cellulose, sodium carboxymethyl cellulose, ethyl or propyl cellulose, polyacrylic acids, polyacrylates, polyvinylpyrrolidones, vinyl pyrrolidone/vinyl acetate copolymers, polyvinyl alcohols, polyethylene oxide polymers, polyacrylamides, polyethylene glycols, gelatines, collagen, alginates, pectin, pullulan, tragacanth, chitosan, alginic acid, arabinogalactan, galactomannan, agar, agarose, carrageenan, and natural gums, wherein polyvinyl alcohols are especially preferred.

The multi-layer oral thin film according to the invention is preferably characterised in that the at least one polymer, preferably the water-soluble polymer, is provided in the active-agent-containing matrix layer of the multi-layer oral thin film in an amount of from 10 to 90 wt. %, preferably from 20 to 60 wt. %, especially preferably from 30 to 50 wt. %, in relation to the total weight of the active-agent-containing matrix layer.

The at least one pharmaceutically active agent is in principle not subject to any restriction, but is preferably selected from all pharmaceutically active agents which are suitable for oral and/or transmucosal application.

According to the present invention, all pharmaceutically acceptable salts and solvates of the particular pharmaceutically active agent are also subsumed under the pharmaceutically active agent.

Preferred active agents are selected from the group comprising the active agent classes of analgesics, hormones, hypnotics, sedatives, antiepiletics, analeptics, psychoneurotropic drugs, neuro-muscle blockers, antspasmodics, antihistamines, antiallergics, cardiotonics, antiarrhythmics, diuretics, hypotensives, vasopressors, antidepressants, antitussives, expectorants, thyroid hormones, sexual hormones, antidiabetics, antitumour active agents, antibiotics, chemotherapeutics and narcotics, although this group is not exhaustive.

The at least one pharmaceutically active agent is especially preferably ketamine and/or a pharmaceutically active salt or solvate thereof, preferably ketamine HCl.

Ketamine is understood to mean (S)-(±)-2-(2-chlorophenyl)-2-(methylamino)cyclohexan-1-one, (R)-(±)-2-(2-chlorophenyl)-2-(methylamino)cyclohexan-1-one, and the racemate (RS)-(±)-2-(2-chlorophenyl)-2-(methylamino)cyclohexan-1-one.

(S)-ketamine or a pharmaceutically acceptable salt thereof, especially (S)-ketamine HCl, is especially preferably present as a single stereoisomer of ketamine, since the analgesic and anaesthetic potency of (S)-ketamine is approximately three times higher than that of the (R) form.

The active agent content in the matrix layer can vary within relatively wide limits. A range of from 10 to 60 wt. %, in relation to the dry weight of the matrix layer, can be stated as suitable. In one embodiment, the proportion of active agent in the matrix layer lies rather in the lower range, for example if the active agent has a strong unpleasant taste, which has to be compensated for by a greater amount of taste-masking agents. In this case, a range of from 10 to 40 wt. % can be stated as suitable active agent fraction. In another embodiment, the proportion of active agent in the dosage form according to the invention lies rather in the upper range, wherein a content of from 40 to 60 wt. % and especially a content of from 45 to 55 wt. % can be stated as being especially preferred.

(S)-ketamine or a pharmaceutically acceptable salt thereof is especially preferably present in the matrix layer in an amount of from 45 to 55 wt. % in relation to the dry weight of the active-agent-containing matrix layer.

The multi-layer oral thin film according to the invention is further preferably characterised in that the matrix layer comprises at least one auxiliary substance selected from the group comprising colouring agents, flavourings, sweeteners, plasticisers, taste-masking agents, emulsifiers, enhancers, pH regulators, humectants, preservatives and/or antioxidants.

Each of these auxiliary substances is preferably contained in the matrix layer in an amount of from 0.1 to 40 wt. %, preferably from 0.1 to 30 wt. %, especially preferably from 0.1 to 15 wt. %, very especially preferably from 0.1 to 10 wt. %, or 0.1 to 5 wt. %, in relation to the total weight of this layer.

The at least one polyethylene glycol preferably has a mean molecular weight of at least 20,000 g/mol to 7,000,000 g/mol, preferably from 40,000 g/mol to 500,000 g/mol, especially preferably from 95,000 g/mol to 105,000 g/mol, especially about 100,000 g/mol.

The molecular weight is derived from the rheology measurements described below.

The multi-layer oral thin film according to the invention is further preferably characterised in that the at least one polyethylene glycol has a viscosity of from 30 mPa s to 50 mPa s, measured at 25° C.

The viscosities stated refer in each case to a 5 wt. % solution of polyethylene glycol in water and are measured on a Brookfield viscometer, model RVF, with spindle no. 1 at 50 rpm and at a temperature of 25° C.

A polyethylene glycol known under the trade name POLYOX WSR N-10 (Dow Chemical) is especially preferred.

The multi-layer oral thin film according to the invention is further preferably characterised in that the at least one polyethylene glycol is contained in the at least one backing layer in an amount of from 60 to 100 wt. %, preferably in an amount of from 80 to 100 wt. %, in relation to the total weight of the at least one backing layer.

The multi-layer oral thin film according to the invention is further preferably characterised in that the at least one polyethylene glycol is contained in the at least one backing layer in an amount of from 65 to 100 wt. %, or 70 to 100 wt. %, or 85 to 100 wt. %, or 90 to 100 wt. %, or 95 to 100 wt. %, in relation to the total weight of the at least one backing layer.

The multi-layer oral thin film according to the invention is further preferably characterised in that the at least one polyethylene glycol is contained in the at least one backing layer in an amount of from 60 to 97.5 wt. %, or 65 to 97.5 wt. %, or 70 to 97.5 wt. %, or 80 to 97.5 wt. %, or 85 to 97.5 wt. %, or 90 to 97.5 wt. %, or 95 to 97.5 wt. %, in relation to the total weight of the at least one backing layer.

The multi-layer oral thin film according to the invention is further preferably characterised in that the at least one polyethylene glycol is contained in the at least one backing layer in an amount of from 60 to 97.5 wt. %, or 65 to 97.5 wt. %, or 70 to 97.5 wt. %, or 80 to 97.5 wt. %, or 85 to 97.5 wt. %, or 90 to 97.5 wt. %, or 95 to 97.5 wt. %, as well as additionally 2 to 2.5 wt. % of at least one plasticiser, preferably glycerol, in relation to the total weight of the at least one backing layer.

The multi-layer oral thin film according to the invention is preferably characterised in that the at least one backing layer comprises at least one auxiliary substance selected from the group comprising colouring agents, flavourings, sweeteners, taste-masking agents, emulsifiers, enhancers, pH regulators, humectants, preservatives and/or antioxidants.

Each of these auxiliary substances is preferably contained in the backing layer in an amount of from 0.1 to 10 wt. %, preferably from 0.1 to 5 wt. %, especially preferably from 0.1 to 2.5 wt. %, in relation to the total weight of this layer.

The multi-layer oral thin film according to the invention is preferably characterised in that the at least one backing layer contains at least one plasticiser, preferably glycerol, preferably in an amount of from 0.5 to 5 wt. %, especially preferably in an amount of from 2 to 2.5 wt. %, in relation to the total weight of the at least one backing layer.

The multi-layer oral thin film according to the invention is, in principle, not limited in the number of layers contained.

For example, embodiments are also conceivable in which the multi-layer oral thin film has several active-agent-containing matrix layers.

Embodiments are also conceivable in which the multi-layer oral thin film has two backing layers on opposite sides of the multi-layer oral thin film.

Especially, at least one adhesive layer can be provided between the at least one matrix layer and the at least one backing layer in order to connect the at least one matrix layer and the at least one backing layer to one another as firmly as possible.

An adhesive layer means a layer that can act as an adhesive as defined in DIN EN 923:2016-03. Accordingly, a non-adhesive layer cannot act as an adhesive as defined above.

Suitable adhesive coatings are, especially, water-soluble adhesive coatings as described in DE 10 2014 127 452 A1, the content of which is hereby incorporated in full.

Suitable adhesive layers comprise at least one water-soluble polymer and at least one plasticiser, wherein the at least one water-soluble polymer in the at least one water-soluble adhesive layer comprises shellac, a vinylpyrrolidone/vinyl acetate copolymer, a polyvinylcaprolactam/polyvinyl acetate/polyethylene glycol copolymer, hydroxypropylcellulose or hydroxypropyl methylcellulose and/or polyvinylpyrrolidone, and wherein the at least one plasticiser in the at least one water-soluble adhesive layer comprises glycerol, polyethylene glycol, especially polyethylene glycol 200, and/or tributyl citrate. The weight ratio of the at least one water-soluble polymer to the at least one plasticiser in the at least one adhesive layer is preferably about 85 to 50 to about 15 to 50.

Such an adhesive layer, which contains at least one water-soluble polymer and at least one plasticiser, can, as an intermediate water-soluble adhesive layer, firmly adhere two further layers, which are not tacky per se, to one another and thus enable the construction of multi-layer oral films without multiple overcoating operations.

The multi-layer oral thin film according to the invention is preferably characterised in that the multi-layer oral thin film consists of exactly two layers, specifically the matrix layer containing at least one polymer and at least one pharmaceutically active agent and a backing layer containing at least one polyethylene glycol.

The active-agent-containing matrix layer can be a smooth film in one embodiment.

The multi-layer oral thin film according to the invention is preferably characterised in that the matrix layer is present in the form of a solidified foam that has voids.

The voids and the associated larger surface area of the films facilitate especially the access of water or saliva or other bodily fluids into the interior of the dosage form and thus accelerate the dissolution of the dosage form and the release of the active agent.

In the case of a rapidly absorbing active agent, transmucosal absorption can also be improved by the rapid dissolution of the matrix layer.

On the other hand, the wall thickness of said voids is preferably small, as these represent solidified bubbles, for example, so that rapid dissolution or destruction of these voids takes place.

A further advantage of this embodiment is that, despite the comparatively high area density, faster drying can be achieved by formulating as a foam than with a comparable non-foamed composition.

The multi-layer oral thin film according to the invention is preferably characterised in that the voids are isolated from one another and are preferably present in the form of bubbles, the voids being filled with air or a gas, preferably with an inert gas, especially preferably with nitrogen, carbon dioxide, helium or a mixture of at least two of these gases.

According to another embodiment, it is provided that the voids are connected to one another, preferably by forming a continuous channel system penetrating the matrix.

Said voids preferably have a volume fraction of from 5 to 98%, preferably from 50 to 80%, in relation to the total volume of the matrix layer. In this way, the advantageous effect of accelerating the dissolution of the matrix layer is influenced favourably.

Furthermore, surface-active agents or surfactants can be added to the matrix polymer or the polymer matrix for foam formation or to the obtained foam before or after drying in order to improve the stability of the foam before or after drying.

Another parameter that influences the properties of the dosage form according to the invention is the diameter of the voids or bubbles. The bubbles or voids are preferably created with the aid of a foam whipping machine, with which the diameter of the bubbles can be adjusted in a wide range, almost arbitrarily. Thus, the mean diameter of the bubbles or voids, determined for example by microtomography or microscopy, can be in the range of from 1 to 350 μm. Especially preferably, the diameter is in the range of 40 and 200 μm.

The multi-layer oral thin film according to the invention preferably has a rough side with an average roughness Ra greater than 2.0 μm and a smooth side with an average roughness Ra less than 1.0 μm.

In another embodiment, the multi-layer oral thin film according to the invention is characterised in that the multi-layer oral thin film has a rough side and a smooth side, wherein the rough side has an average roughness Ra of 1.0 μm more than the smooth side.

The roughness can be measured here for example on a KLA Tencor P15 profilometer with a measuring stylus of 2 μm radius. To this end, for example, both sides of an oral thin film are surface-scanned over an area of 2 mm×2 mm. 3 line scans are selected randomly from the scan field of 2 mm×2 mm and the roughness parameters Ra, Rq, Rp and Rv are determined.

The following roughness parameters can be determined for assessment of the sample:
Ra: average roughness; specifies the average distance of a measurement point from the centre line.
Rq: square roughness (also RMS roughness); is calculated from the average of the deviation squares and corresponds to the square average.
Rp: peak height, distance between the centre line and the greatest measured value.
Rv: valley depth, distance between the centre line and the smallest measured value.

The multi-layer oral thin film according to the invention is further preferably characterised in that the multi-layer oral thin film has an at least 30% slower release of the at least one pharmaceutically active agent than the matrix layer alone without backing layer.

This means that the presence of the backing layer on the matrix layer can slow the release of the at least one pharmaceutically active agent by at least 30%.

The oral thin film according to the invention preferably has an area of from 0.5 cm² to 10 cm², especially preferably from 1 cm² to 8 cm².

The oral thin film according to the invention is preferably characterised in that the area density of the multi-layer oral thin film is 10 to 500 g/m², preferably 70 to 400 g/m².

The area density of the matrix layer is preferably at least 10 g/m², more preferably at least 20 g/m², or at least 30 g/m², or most preferably at least 50 g/m², or less than or equal to 400 g/m², more preferably less than or equal to 350 g/m², or less than or equal to 300 g/m², or most preferably less than 150 g/m². Preferably, the area density is 10 to 400 g/m², more preferably 20 to 350 g/m², or 30 to 300 g/m², most preferably 50 to 150 g/m².

The area density of the backing layer is important for controlling the dissolution behaviour and the function of the backing layer in order to protect the active agent from dissolving in saliva. A certain thickness is expedient in order to ensure sufficient protection of the active agent and also a sufficient dissolution time, which should normally be at least as long as the active permeation requirement and thus the dissolution time of the matrix layer.

It is therefore preferred that the backing layer has an area density of at least 10 g/m², more preferably at least 20 g/m² or at least 30 g/m², most preferably at least 50 g/m² or an area density of less than or equal to 400 g/m², more preferably less than or equal to 350 g/m², or less than or equal to 300 g/m², or most preferably less than or equal to 150 g/m², or an area density of from 10 to 400 g/m², more preferably from 20 to 350 g/m², or from 30 to 300 g/m², most preferably from 50 to 150 g/m².

Preferably, the matrix layer and the at least one backing layer each have a layer thickness of from about 10 μm to about 500 μm, especially preferably from about 20 μm to about 300 μm.

Especially, the backing layer may be equal to or larger than the matrix layer in terms of size. Thus, in certain embodiments the size of the backing layer and the size of the active-agent-containing matrix layer are the same, while in other embodiments the backing layer is larger than the surface area of the matrix layer. While a layered structure with the same size of backing layer and matrix layer is easier to produce, since a two-layer sheet can be punched out to provide the layered structure, a layered structure with a backing layer larger than the matrix layer is more difficult to produce, but also offers the advantage that there is less risk of active leakage, since the edge of the matrix layer is also covered by the carrier layer.

The present invention also relates to a method for producing the multi-layer oral thin film according to the invention, the method comprising the steps of:
a) producing and spreading a solution or suspension comprising the at least one polyethylene glycol, and then drying the spread solution or suspension in order to obtain a film comprising the at least one polyethylene glycol,
b) producing a solution, dispersion or melt which at least comprises the at least one polymer and the at least one pharmaceutical active agent,
b1) optionally foaming the solution, dispersion or melt from step b) by introducing a gas or gas mixture by chemical gas generation or by expansion of a dissolved gas,
c) spreading the solution, dispersion or melt from step b) or the optionally foamed solution, dispersion or melt from step b1) onto the film obtained in step a) comprising the at least one polyethylene glycol in order to obtain a composite,
d) drying the composite obtained in step c) in order to obtain a multi-layer oral thin film.

Steps a) and b) can be carried out in any order.

It is clear to a person skilled in the art that step b1) is then only necessary if the active-agent-containing matrix layer is to be present in the form of a solidified foam that has voids.

In step c), further layers, such as an adhesive layer, may also be present on the matrix layer.

The joining together of the two films from step a) and b) can be done in principle by methods known to a person skilled in the art. For example, another film can be applied to a first film by means of coating; it is in principle irrelevant which film is coated on which film, provided the backing layer forms one of the outermost layers. Furthermore, the two layers can be connected to one another by an adhesive layer as described above.

The layers are preferably connected to one another by means of heat. For this purpose, the drying in step d) is performed at a temperature that is higher than the melting temperature of the matrix layer and/or the backing layer. As a result of this, the matrix layer and/or the backing layer starts to melt for example and thus ensures a firm connection of the two layers in the event of subsequent cooling.

Suitable temperatures are 40 to 100° C., preferably 55 to 80° C. Drying is preferably performed for a period of from 10 to 60 min.

In a further embodiment, the method according to the invention is characterised in that the above-mentioned steps c) and d) are replaced by the steps
c2) spreading the solution, dispersion or melt from step b) or the optionally foamed solution, dispersion or melt from step b1) in order to obtain a film comprising the at least one polymer and the at least one pharmaceutical active agent, and
d2) connecting the films obtained in step a) and c2), preferably by lamination by exposure to heat above the melting point of one of the polymers contained in the films in order to obtain a multi-layer oral thin film.

By way of the modified method, embodiments of the multi-layer oral thin film in which both sides of the active-agent-containing matrix layer are covered by a layer comprising at least one polyethylene glycol can also be easily produced. Such multi-layer oral thin films dissolve very slowly.

The present invention further relates to a multi-layer oral thin film obtainable by the method described above.

The present invention additionally relates to a multi-layer oral thin film as described above, or obtainable by the method described above, as a medicament.

The present invention additionally relates to a multi-layer oral thin film, as described above or obtainable by the method described above, wherein ketamine, preferably S-ketamine, is used as pharmaceutically active agent, for use in the treatment of pain and/or depression, especially to reduce the risk of suicide and/or for use as a general anaesthetic, preferably to initiate and carry out general anaesthesia, or as a supplement in the case of local anaesthesia and/or as an analgesic.

The preferred embodiments described above for the multi-layer oral thin film according to the invention are also applicable for the method according to the invention, the multi-layer oral thin film obtained by this method, and use thereof as a medicament.

The invention will be explained in greater detail hereinafter on the basis of non-limiting examples.

### EXAMPLE 1

Example formulation formed of a polyethylene glycol (polyox) backing layer and an S-ketamine-containing matrix layer in the form of a foam.

TABLE 1

| | |
|---|---|
| Material | Amount [wt. %] |

Active-agent-containing matrix layer

| | |
|---|---|
| S-ketamine HCl | 50.00 |
| PVA 4-88 | 41.70 |
| Saccharin Na | 1.00 |
| Sucralose | 2.00 |
| Glycerol | 2.30 |
| Flavouring 1 | 1.00 |
| Flavouring 2 | 2.00 |
| Process solvent | Water |

Backing layer

| | |
|---|---|
| Polyox WSR N10 | 97.50 |
| Glycerol | 2.30 |
| Colouring agent | 0.20 |
| Process solvent | Water |
| Area density (dry) | Active-agent-containing matrix layer: |
| | 118.7 g/m² |
| | Backing layer: 100 g/m² |
| Coating weight | Active-agent-containing matrix layer: |
| (incl. residual water) | 121.9 g/m² |
| | Backing layer: 101.5 g/m² |
| S-ketamine base in | 14.9 mg/2.9 cm² |
| mg/oral thin film | Corresponds to 17.2 mg S-ketamine |
| | HCl |

PVA 4-88: Polyvinyl alcohol
Polyox WSR N10: Polyethylene glycol with a MW of 100,000 g/mol from Dow Chemical

The multi-layer oral thin film according to the above composition was produced as follows.

The raw materials of the backing layer are processed by known weighing and stirring techniques and using suitable stirring tools and motors to give a homogeneous mass. This is then coated with the aid of coating tools (for example film applicators) onto a web-like substrate (having a certain area density). The moist film is dried with the aid of a drying cabinet at 70° C. for 15 min.

The raw materials of the active-agent-containing matrix layer are likewise processed by known weighing and stirring techniques and also using suitable stirring tools and motors to give a homogeneous mass. This mass is in turn foamed using stirring techniques and the resultant foam is coated onto the dry backing layer. This is done, again, with the aid of coating tools (for example film applicators). The obtained film, consisting of backing layer and matrix layer, is dried with the aid of a drying cabinet once again at 70° C. for 15 min.

This multi-layer film can be converted, after drying, into the form of smaller oral thin films.

### EXAMPLE 2

Example formulation formed of a polyethylene glycol (polyox) backing layer and an S-ketamine-containing matrix layer in the form of a foam, the layers being produced individually and then connected to one another by lamination.

TABLE 2

| | Amount |
|---|---|
| | [wt. %] |
| Active-agent-containing matrix layer | |
| (S)-ketamine HCl | 50.00% |
| PVA 4-88 | 41.70% |
| Saccharin Na | 1.00% |
| Sucralose | 2.00% |
| Glycerol | 2.30% |
| Flavouring 1 | 1.00% |
| Flavouring 2 | 2.00% |
| Process solvent | Purified water |
| Area density (dry) | 117.3 g/m² |
| Area density (incl. res. solvent) | 121.9 g/m² |
| Backing layer | |
| Polyox WSR N10 | 97.50% |
| Glycerol | 2.3% |
| Colouring agent | 0.20% |
| Process solvent | Purified water |
| Area density (dry) | 98.2 g/m² |
| Area density (incl. res. solvent) | 101.5 g/m² |

The raw materials of the backing layer are processed by known weighing and stirring techniques and using suitable stirring tools and motors to give a homogeneous mass. This is then coated with the aid of coating tools (for example film applicators) onto a web-like substrate (having a certain area density). The moist film is dried with the aid of a drying cabinet at 70° C. for 15 min.

The raw materials of the active-agent-containing matrix layer are likewise processed by known weighing and stirring techniques and also using suitable stirring tools and motors to give a homogeneous mass. This mass is in turn foamed using stirring techniques and the resultant foam is coated onto a web-like substrate. This is done, again, with the aid of coating tools (for example film applicators). The moist film is dried with the aid of a drying cabinet at 70° C. for 15 min.

The two layers were joined together by lamination. To this end, the backing layer was heated temporarily to 70° C. and then connected to the active-agent-containing matrix layer by lamination.

This multi-layer film can be converted into the form of smaller oral thin films.

In this multi-layer oral thin film, an active-agent-free polyox layer serves as backing layer for the actual active-agent-containing matrix layer of the oral thin film. The protective layer can mask the taste of the active agent and can avoid rapid swallowing of the active agent and thus can increase the oromucosal bioavailability. Specifically, the oral thin film according to the invention is distinguished in that the active agent streak can be coated directly onto the polyox layer during the production process.

By drying above the melting point of polyox, active-agent-containing matrix layer and the backing layer combine to form a firm bond. There is thus no need for an adhesive layer.

Furthermore, the patient can identify, on the basis of the different roughness of the layers (the polyox-containing backing layer is very smooth or film-like and the active-agent-containing matrix layer in the form of a foam is very rough), which side of the OTF is to be applied to the mucosa and whether the correct side has also been applied in this way (different mouthfeel depending on which layer is at the top or bottom).

Disintegration Time Properties:

The disintegration time of a multi-layer oral thin film (with backing layer) with a composition according to Table 2 was compared with the disintegration time of a one-layer oral thin film with a composition according to Table 3 (without backing layer). Production was performed similarly to the above-described method for the oral thin film (with backing layer) with a composition according to Table 2.

TABLE 3

| | Amount |
|---|---|
| Active-agent-containing matrix layer | [wt. %] |
| (S)-ketamine HCl | 50.00% |
| PVA 4-88 | 41.70% |
| Saccharin Na | 1.00% |
| Sucralose | 2.00% |
| Glycerol | 2.30% |
| Flavouring 1 | 1.00% |
| Flavouring 2 | 2.00% |
| Process solvent | Purified water |
| Area density (dry) | 117.3 g/m² |
| Area density (incl. res. solvent) | 121.9 g/m² |

The disintegration times of the oral thin films were measured in accordance with USP 701 with a tablet disintegration device (Pharma-Test DIST-3 Triple Basket Tablet Disintegration Tester, 30 strokes per minute over a distance of 55 mm, in 11 pH 6.8 phosphate buffer). Oral thin films with a size of 2.72 cm² were placed in a basket (sinker) and positioned in a glass tube secured to the instrument. Lastly, the time until only residues of the oral thin films were still present in the basket was determined.

TABLE 4

| Oral thin film | Disintegration time |
|---|---|
| only active-agent-containing | 3 s |
| matrix layer (Table 3) | |
| active-agent-containing matrix | 44 s |
| layer with backing layer (Table 2) | |

Active Agent Release:

The in vitro release of the active agent from the oral thin films with the compositions according to Tables 2 and 3 was also determined as follows:

Release Method:

With the in vitro release, S-ketamine is released from S-ketamine HCl oral thin films (OTF) and is determined. The active agent is released in phosphate buffer pH 6.8 USP and is then determined by a gradient reverse phase HPLC method. The quantification was performed against an external standard.

The release is performed with Dissolution Apparatus 2—(Paddle over sinker) according to USP <711>.

| Sinker: | Stainless Steel Capsule Sinker with | |
|---|---|---|
| | 10 Spirals, 31.0 × 11.0 mm | |
| | Capacity (Sotax Style) | |
| Stirring speed: | 50 | rpm |
| Distance between the vessel base | 25 mm ± 2 mm | |
| and the lower edge of the paddle: | | |
| Temperature: | 37° C. ± 0.5° C. | |
| Release medium: | Phosphate buffer pH 6.8 USP | |
| Release medium volume: | 900 | mL |
| Sample measurement times: | 1, 3, 5, 10 and 15 min | |
| Sample volume: | 5.0 | mL |

TABLE 5

| | | only active-agent- | active-agent-containing |
|---|---|---|---|
| in vitro release | | containing matrix layer | matrix layer with |
| [% of LC*] | | (Table 3) | backing layer (Table 2) |
| after 1 | Mean | 101 | 60 |
| min | Min | 96 | 47 |
| | Max | 106 | 75 |
| | RSD | 4.2 | 19.9 |
| | [%] | | |
| after 3 | Mean | 103 | 102 |
| min | Min | 100 | 97 |
| | Max | 108 | 111 |
| | RSD | 3.6 | 4.7 |
| | [%] | | |
| after 5 | Mean | 104 | 104 |
| min | Min | 100 | 100 |
| | Max | 109 | 105 |
| | RSD | 4.0 | 1.9 |
| | [%] | | |
| after | Mean | 104 | 103 |
| 10 min | Min | 100 | 101 |
| | Max | 109 | 105 |
| | RSD | 3.9 | 1.4 |
| | [%] | | |
| after | Mean | 103 | 104 |
| 15 min | Min | 100 | 101 |
| | Max | 108 | 106 |
| | RSD | 3.9 | 1.9 |
| | [%] | | |

*Label Claim = specified content

### EXAMPLE 3

A multi-layer oral thin film with a composition according to Table 6 was produced, wherein an active-agent-containing matrix layer is covered from both sides with a backing layer.

TABLE 6

| | |
|---|---|
| Material | Amount [wt. %] |

Active-agent-containing matrix layer

| | |
|---|---|
| S-ketamine HCl | 50.00 |
| PVA 4-88 | 41.70 |
| Saccharin Na | 1.00 |
| Sucralose | 2.00 |
| Glycerol | 2.30 |
| Flavouring 1 | 1.00 |
| Flavouring 2 | 2.00 |
| Process solvent | Water |

2 × backing layer (in each case)

| | |
|---|---|
| Polyox WSR N10 | 97.50 |
| Glycerol | 2.30 |
| Colouring agent | 0.20 |
| Process solvent | Water |
| Area density (dry) | Active-agent-containing matrix layer: |
| | 118.7 g/m² |
| | Backing layer: 100 g/m² |
| Coating weight | Active-agent-containing matrix layer: |
| (incl. residual water) | 121.9 g/m² |
| | Backing layer: 101.5 g/m² |
| S-ketamine base in mg/oral | 14 mg/2.72 cm² |
| thin film | Corresponds to 16.1 mg S-ketamine |
| | HCl |

The raw materials of the two backing layers are processed by known weighing and stirring techniques and using suitable stirring tools and motors to give a homogeneous mass. This is then coated with the aid of coating tools (for example film applicators) onto a web-like substrate (having a certain area density). The moist film is dried with the aid of a drying cabinet at 70° C. for 15 min.

The raw materials of the active-agent-containing matrix layer are likewise processed by known weighing and stirring techniques and also using suitable stirring tools and motors to give a homogeneous mass. This mass is in turn foamed using stirring techniques and the resultant foam is coated onto a web-like substrate. This is done, again, with the aid of coating tools (for example film applicators). The moist film is dried with the aid of a drying cabinet at 70° C. for 15 min.

The two backing layers and the intermediate active agent layer were joined together by lamination. To this end, a backing layer was heated temporarily to 70° C. and then connected to the active-agent-containing matrix layer by lamination. A second backing layer was then heated temporarily to 70° C. and then laminated onto the free side of the active-agent-containing matrix layer of the previous laminate. A laminate of composition backing layer—active-agent-containing matrix layer—backing layer was thus obtained.

The obtained laminate can be converted into the form of smaller oral thin films.

### EXAMPLE 4

A multi-layer oral thin film with a composition according to Table 7 was produced similarly to Example 2. The active-agent-containing matrix layer was not foamed in this case.

TABLE 7

| | Amount |
|---|---|
| | [wt. %] |
| Backing layer | |
| Polyox WSR N10 | 97.50% |
| Glycerol | 2.3% |
| Colouring agent | 0.20% |
| Process solvent | Purified water |
| Area density (dry) | 98.2 g/m² |
| Area density (incl. res. process solvent) | 101.5 g/m² |
| Active-agent-containing matrix layer | |
| HPMC 2910 (603) | 39.5 |
| HPMC 2910 (60SH50) | 10.0 |
| S-ketamine | 41.0 |
| Saccharin Na | 2.0 |
| Sucralose | 1.0 |
| Glycerol | 3.5 |
| Flavouring | 3.0 |
| Process solvent | Purified water |
| Area density (dry) | 175 g/m² |
| Area density (incl. res. process solvent) | 184.5 g/m² |
| Note | not foamed |

HPMC 2910 (603): Hydroxypropylmethylcellulose 2910 (603)
HPMC 2910 (60SH50): Hydroxypropylmethylcellulose 2910 (60SH50)

The used HPMCs differ in the viscosity of a 2% aqueous solution: 603: 3 mPaS vs. 60SH50: 50 mPaS.

The two layers were produced separately and connected to one another by lamination by heating to 70° C. After cooling, the two layers were firmly connected.

### EXAMPLE 5

TA morphological surface examination was performed on a KLA Tencor P15 profilometer with a measuring stylus of 2 μm radius. Both sides of an OTF from Example 1 were surface-scanned over an area of 2 mm×2 mm. 3 line scans were selected randomly from the scan field of 2 mm×2 mm and the roughness parameters
Ra, Rq, Rp and Rv were determined.

The following roughness parameters were determined for assessment of the sample:
Ra: average roughness; specifies the average distance of a measurement point from the centre line.
Rq: square roughness (also RMS roughness); is calculated from the average of the deviation squares and corresponds to the square average.
Rp: peak height, distance between the centre line and the greatest measured value.
Rv: valley depth, distance between the centre line and the smallest measured value.

The roughness parameters show that the OTF has two different rough sides. The side of the backing layer is very smooth, comparable to smooth materials such as polished steel. By contrast, the side of the matrix-containing active agent layer is much rougher. On account of the different roughness of the two OTF sides, a patient can identify, by feeling, which side is the backing layer and which side is the active-agent-containing side. For example, correct positioning of the OTF in the mouth can thus be identified (for example rough side at the bottom towards the oral mucosa, smooth backing layer at the top).

OTF on the Backing Layer Side:

| Measurement | Ra/μm | Rq/μm | Rp/μm | Rv/μm |
|---|---|---|---|---|
| 1 | 0.76 | 1.06 | 4.85 | 1.40 |
| 2 | 0.55 | 0.86 | 5.54 | 0.87 |
| 3 | 0.74 | 1.15 | 6.64 | 2.09 |
| Mean value | 0.68 | 1.02 | 5.68 | 1.45 |

OTF on the Active-Agent-Con a n Ng Matrix Layer Side:

| Measurement | Ra/μm | Rq/μm | Rp/μm | Rv/μm |
|---|---|---|---|---|
| 1 | 3.17 | 3.89 | 11.64 | 11.15 |
| 2 | 2.73 | 3.84 | 11.59 | 19.96 |
| 3 | 3.84 | 4.74 | 10.61 | 12.07 |
| Mean value | 3.24 | 4.16 | 11.28 | 14.39 |

### EXAMPLE 6

Further release measurements with profile dissolution and a higher temporal dissolution then in Table 5 were performed.

The following were compared:
active-agent-containing matrix layer (Table 3)
active-agent-containing matrix layer with backing layer on one side (Table 2)
active-agent-containing matrix layer with backing layer on both sides (Table 7)

To this end, S-ketamine is released from S-ketamine HCl oral thin films (OTF) and is determined. The active agent is released in phosphate buffer pH 6.8 USP and is then determined by a gradient reverse phase HPLC method. The quantification was performed against an external standard.

The release is performed with Dissolution Apparatus 2—(Paddle over sinker) according to USP <711>.

| Sinker: | Stainless Steel Capsule Sinker with | |
|---|---|---|
| | 10 Spirals, 31.0 × 11.0 mm | |
| | Capacity (Sotax Style) | |
| Stirring speed: | 50 | rpm |
| Distance between the vessel base | 25 mm ± 2 mm | |
| and the lower edge of the paddle: | | |
| Temperature: | 37° C. ± 0.5° C. | |
| Release medium: | Phosphate buffer pH 6.8 USP | |
| Release medium volume: | 900 | mL |
| Sample measurement times: | 1, 3, 5, 10 and 15 min | |
| Sample volume: | 5.0 | mL |

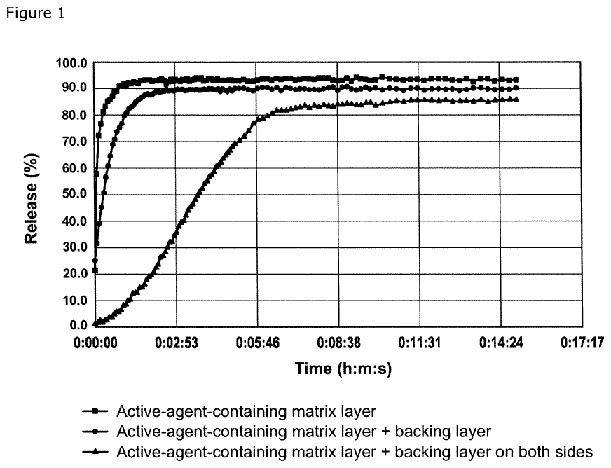

The results are shown in FIG. 1. A person skilled in the art can see that the release profile of the matrix layer can be varied with the backing layer.

1. A multi-layer oral thin film comprising a matrix layer, which contains at least one polymer and at least one pharmaceutically active agent, and at least one backing layer, wherein the at least one backing layer comprises at least one polyethylene glycol in an amount of from 60 to 100 wt. %. in relation to the total weight of the at least one backing layer.

2. The multi-layer oral thin film according to claim 1, wherein the matrix layer comprises at least one water-soluble polymer.

3. The multi-layer oral thin film according to claim 2, wherein the at least one water-soluble polymer is selected from the group comprising starch and starch derivatives, dextrans, cellulose derivatives, such as carboxymethyl cellulose, hydroxypropyl cellulose, hydroxyethyl cellulose, hydroxypropyl methylcellulose, hydroxypropyl ethyl cellulose, sodium carboxymethyl cellulose, ethyl or propyl cellulose, polyacrylic acids, polyacrylates, polyvinylpyrrolidones, vinyl pyrrolidone/vinyl acetate copolymers, polyvinyl alcohols, polyethylene oxide polymers, polyacrylamides, polyethylene glycols, gelatines, collagen, alginates, pectin, pullulan, tragacanth, chitosan, alginic acid, arabinogalactan, galactomannan, agar, agarose, carrageenan, and natural gums.

4. The multi-layer oral thin film according to claim 1, wherein the at least one pharmaceutically active agent is selected from the group comprising the active agent classes of analgesics, hormones, hypnotics, sedatives, antiepiletics, analeptics, psychoneurotropic drugs, neuro-muscle blockers, antspasmodics, antihistamines, antiallergics, cardiotonics, antiarrhythmics, diuretics, hypotensives, vasopressors, antidepressants, antitussives, expectorants, thyroid hormones, sexual hormones, antidiabetics, antitumour active agents, antibiotics, chemotherapeutics and narcotics, wherein the at least one pharmaceutically active agent preferably comprises ketamine, especially preferably (S)-ketamine.

5. The multi-layer oral thin film according to claim 1, wherein the matrix layer further comprises at least one auxiliary substance selected from the group comprising colouring agents, flavourings, sweeteners, plasticisers, taste-masking agents, emulsifiers, enhancers, pH regulators, humectants, preservatives and/or antioxidants.

6. The multi-layer oral thin film according to claim 1, wherein the at least one polyethylene glycol has a mean molecular weight of from 20,000 g/mol to 7,000,000 g/mol, preferably from 40,000 g/mol to 500,000 g/mol, especially preferably from 95,000 g/mol to 105,000 g/mol, especially of about 100,000 g/mol.

7. The multi-layer oral thin film according to claim 1, wherein the at least one polyethylene glycol has a viscosity of from 30 mPa s to 50 mPa s, measured in 5 wt. % aqueous solution at 25° C.

8. The multi-layer oral thin film according to claim 1, wherein the at least one polyethylene glycol is preferably contained in the at least one backing layer in an amount of from 60 to 100 wt. %, preferably in an amount of from 80 to 100 wt. %, in relation to the total weight of the at least one backing layer.

9. The multi-layer oral thin film according to claim 1, wherein the at least one backing layer contains at least one plasticiser, preferably glycerol, preferably in an amount of from 0.5 to 5 wt. % in relation to the total weight of the at least one backing layer.

10. The multi-layer oral thin film according to claim 1, wherein the multi-layer oral thin film consists of exactly two layers, specifically the matrix layer containing at least one polymer and at least one pharmaceutically active agent and the backing layer containing at least one polyethylene glycol.

11. The multi-layer oral thin film according to claim 1, wherein the matrix layer is present in the form of a solidified foam that has voids.

12. The multi-layer oral thin film according to claim 11, wherein the voids are isolated from one another and are preferably present in the form of bubbles, wherein the voids are filled with air or a gas, preferably with an inert gas, especially preferably with nitrogen, carbon dioxide, helium or a mixture of at least two of these gases.

13. The multi-layer oral thin film according to claim 11, wherein the voids are connected to one another and preferably form a channel system penetrating the matrix layer.

14. The multi-layer oral thin film according to claim 11, wherein the voids in the matrix layer account for a volume fraction of from 5 to 98%, preferably from 50 to 80%, in relation to the total volume of the layer in question.

15. The multi-layer oral thin film according to claim 1, wherein the oral thin film has a rough side with an average roughness Ra greater than 2.0 μm and a smooth side with an average roughness Ra less than 1.0 μm.

16. The multi-layer oral thin film according to claim 1, wherein the oral thin film has a rough side and a smooth side, wherein the rough side has an average roughness Ra of 1.0 μm more than the smooth side.

17. The multi-layer oral thin film according to claim 1, wherein the multi-layer oral thin film has an at least 30% slower release of the at least one pharmaceutically active agent than the matrix layer alone without backing layer.

18. A method for producing a multi-layer oral thin film according to claim 1, comprising the steps of:
a) producing and spreading a solution or suspension comprising the at least one polyethylene glycol, and then drying the spread solution or suspension in order to obtain a film comprising the at least one polyethylene glycol,
b) producing a solution, dispersion or melt which at least comprises the at least one polymer and the at least one pharmaceutical active agent,
b1) optionally foaming the solution, dispersion or melt from step b) by introducing a gas or gas mixture by chemical gas generation or by expansion of a dissolved gas,
c) spreading the solution, dispersion or melt from step b) or the optionally foamed solution, dispersion or melt from step b1) onto the film obtained in step a) comprising the at least one polyethylene glycol in order to obtain a composite,
d) drying the composite obtained in step c) in order to obtain a multi-layer oral thin film.

19. The method according to claim 18, characterised in that steps c) and d) are replaced by the following steps:
c2) spreading the solution, dispersion or melt from step b) or the optionally foamed solution, dispersion or melt from step b1) in order to obtain a film comprising the at least one polymer and the at least one pharmaceutical active agent,
d2) connecting the films obtained in step a) and c2), preferably by lamination by exposure to heat above the melting point of one of the polymers contained in the films in order to obtain a multi-layer oral thin film.

20. (canceled)

21. A method of administering a medicament comprising providing the multi layer oral thin film of claim 1 to a subject.
